(19) United States
(12) Patent Application Publication
(10) Pub. No.: US 2008/0082030 A1
(43) Pub. Date: Apr. 3, 2008

(54) ADJUSTABLE NOSE SPLINT

(76) Inventor: Steven Mark Clark, Beverly Hills, CA (US)

Correspondence Address: MARC E. HANKIN, ESQ., 11414 THURSTON CIRCLE, LOS ANGELES, CA, 90049, US

(21) Appl. No.: 11/530,371

(22) Filed: Sep. 8, 2006

(51) Int. Cl. A61F 5/00; A61F 5/08; A61F 9/00

(52) U.S. Cl. 602/6; 128/858; 606/204.45

(57) ABSTRACT

Various embodiments of the invention disclose adjustable nose splints that may be used during the rhinoplasty recovery process in order to apply pressure to the patient's nose.

### FIELD OF INVENTION

Various embodiments of this invention relate, generally, to splints; more particularly to adjustable splints that may be used to support the human nose.

### BACKGROUND

Plastic surgeons perform surgery on the nose (rhinoplasty) in order to reshape the nose for cosmetic or physiological reasons, such as improving breathing. Once reserved almost exclusively to rehabilitate those that had suffered an unfortunate accident, plastic surgery is now a popular method for altering and improving one's physiognomy. Among the available cosmetic surgery procedures, rhinoplasty is the most common in the United States, with more than 500,000 people receiving the procedure each year.

Rhinoplasty is often used to increase or decrease the size or reshape the bridge of the nose. In reshaping the bridge of the nose, the surgeon is often required to break the nasal bone in order to decrease its width and give the appearance of a more cosmetically appealing nose. During the recovery period, which lasts up to six weeks, lateral pressure must be maintained on the nasal bone in order to ensure proper healing.

Oftentimes, in order to maintain the pressure, doctors request that patients use their index fingers to apply pressure to the nose. However, doing so is burdensome and does not allow uniform pressure to be applied to the nose over a long period of time. Thus, the recovery may be impeded.

Meanwhile, splints have been developed that are directed towards maintaining the position of bones and assisting in the recovery and healing process. U.S. Pat. Nos. 4,213,452, 4,274,402, and 5,947,123 to Shippert, for example, disclose nose splints that feature firm but malleable structures for supporting the bones of the nose and preventing edema fluid from splaying the nasal bones during recovery. Shippert's nose splints attach to the nose by adhesive. Thus, the amount of pressure that they can provide to the nasal bones is somewhat limited.

Another such device is disclosed by U.S. Pat. No. 5,022,389 to Brennan, which discloses a 3-part nasal splint device. Brennan's device improves upon the basic nasal splint design by allowing malleable metal layer to be adjusted at the lateral wing portions of the splint in order to provide greater lateral pressure to the nose. Brennan's device, however, suffers from some of the same shortcomings because the device similarly relies upon adhesive means to attach to the patient's nose.

Other devices have sought to improve upon the attachment mechanism, such that greater pressure may be placed on the nose as it heals. U.S. Pat. No. 5,817,039 to Raung, for example discloses a nasal splint system that features adhesive tapes which may attach to the forehead and cheeks of the patient, thus creating a stronger grip and increased pressure to the healing nose. U.S. Pat. No. 3,742,743 to Malmin discloses a means for increasing pressure to the nose by using an elastic head-strap that attaches to the nose splint. All of these devices, however, have a limited degree of adjustability, which limits their ability to be customized to the patient's nose for comfort and functionality.

U.S. Pat. No. 2,398,073 to Bonde discloses a device that is directed towards increasing the adjustability of the nose splint. Bonde's patent discloses a nose splint comprised of two horizontally opposed splint sections, the distance between being adjustable by a screw and nut. Bonde's nose splint may be attached to the face of the wearer by adhesive tape that is attached to the cheeks of the wearer. While the width between the lateral splints in Bonde's device is adjustable, the pressure with which the splints are applied to the nose is not. A splint whose pressure system is adjustable is highly desirable in the art because it allows for a more comfortable and functional nose splint during the patient's recovery.

Thus, there remains a long felt need in the art for an adjustable nose splint that uses a comfortable and adjustable system to allow the wearer to adjust the pressure that is applied to the recovering nose.

### SUMMARY OF THE INVENTION

Various embodiments of the invention are directed towards overcoming the above shortcomings by disclosing an adjustable nose splint that uses an extendible clamp system and allows for the pressure applied to the nose to be adjusted by the user, such that the user may configure pressure so that the nose remains comfortable as pressure is applied to the healing nose.

In various embodiments of the invention, the invention features two horizontally opposed lateral splint sections, which connect at a hinge that runs along the uppermost portion of each lateral splint. When the lateral splint sections are displaced relative to one another, a roughly pyramidal shape is formed, so the lateral splint sections approximate the roughly pyramidal shape of the nose. Thus, as the lateral splint sections are compressed towards one another, the sidewalls of the wearer's nose are likewise compressed.

The lateral splint sections are shaped to generally conform to the shape of the human nose. In various embodiments of the invention, the lateral splint sections are further shaped in order to conform to the shape of the alar-sidewall, the supra-alar crease, the alar-facial groove, and other features of the nose's anatomy, by featuring concave edges. The lateral splint sections may also feature curved sections at their upper regions adjacent to the hinge such that the lateral splint sections better conform to the shape of the nasal bone.

The two lateral splint sections, when placed on the nose, are generally parallel and lie adjacent to the sidewalls of the nose. The two lateral splint sections are connected to one another by a hinge. The hinge allows the angle between the lateral splint sections to be adjusted by the user. The hinge may be constructed from the same material as that of the lateral splint sections or any of the known materials in the art.

In order to apply pressure to the nose and attach the adjustable nose splint to the nose, the adjustable nose splint further features a spring. The spring is configured to generally force the narrowing of the angle between the lateral splints of the adjustable nose splint. In various embodiments of the invention, the spring is constructed in a U-shape and attached to the lateral splint sections. The spring may be fixedly or removably attached to the lateral splints. In various embodiments of the invention, the spring is removably attached to the lateral splint sections by projections or tangs that engage the spring. The spring may be constructed from metallic, plastic, rubber, or other such extendable materials known within the art.

So that the pressure applied to the nose may be adjusted by the user, the adjustable nose splint further features a mechanism by which the pressure of the spring may be adjusted. In various embodiments of the invention, the adjustable system comprises a combination of a threaded stud and a knurled nut. The threaded stud or studs are attached to one or more ends of the spring. The threaded stud or studs engage with a knurled nut that can adjust the tension in the spring by regulating the degree of engagement with the stud or studs. In this manner, the user is able to select the amount of pressure applied to the nose by turning the threaded stud. In various embodiments of the invention, the studs feature extra fine threading, such that the increments of tension in the spring may be minutely adjusted by the user. It remains within the contemplation of the invention that the spring may be located over or below the lateral splint sections and adjacent to the wearer's columella.

Thus, the adjustable nose splint may be worn as a cover to the nose with the hinge of the device covering the nasal bone, the lateral splint sections covering the sidewalls of the nose, and the spring pressuring the two lateral splint sections towards one another. The degree of pressure applied to the nose may be adjusted by the wearer or the physician by turning one or more of the knurled nuts. Once in place and suitably adjusted, the adjustable nose splint allows the user to maintain suitable pressure against the exterior of the nose while maintaining the alignment of the nose during the healing process.

In various embodiments of the invention, the adjustable nose splint features further accessories so that it may better carry out its functions. For example, in various embodiments of the invention, the interior lining of the lateral splint sections may feature foam or other compressible material, such that the comfort to the wearer is increased. In various embodiments of the invention, this material may be visco-elastic, such that the material maintains its shape and thereby allows increased comfort under constant pressure. The foam or other compressible material may be fixedly attached or removably attached to the adjustable nose splint, for example, by using adhesives.

It remains within the contemplation of the invention that it may be used with varying materials and with various accessories that are known in the art.

### BRIEF DESCRIPTION OF THE DRAWINGS

FIG. 1 is an illustration of an exploded view showing the parts of the adjustable nose splint in one embodiment of the invention.

### DETAILED DESCRIPTION OF THE DRAWINGS

In the following detailed description of various embodiments of the invention, numerous specific details are set forth in order to provide a thorough understanding of various aspects of one or more embodiments of the invention. However, one or more embodiments of the invention may be practiced without these specific details. In other instances, well-known methods, procedures, and/or components have not been described in detail so as not to unnecessarily obscure aspects of embodiments of the invention.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive. Also, the reference or non-reference to a particular embodiment of the invention shall not be interpreted to limit the scope the invention.

In the following description, certain terminology is used to describe certain features of one or more embodiments of the invention. For instance, “splint” refers to any of the rigid or semi-rigid devices used to prevent motion of a part of the body; “hinge” refers to any of the various jointed or flexible devices that allow for the turning or pivoting of the lateral splints; and “spring” refers to any of the various elastic devices, such as a wire, rubber, or plastic, that regains its original shape after being compressed or extended.

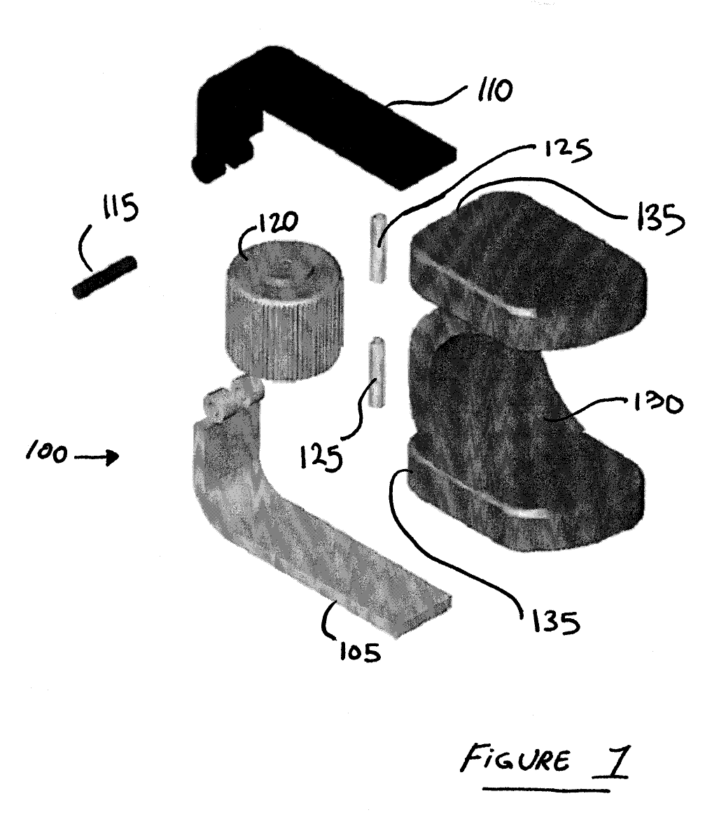

FIG. 1 is an illustration of an exploded view showing the parts of the adjustable nose splint in one embodiment of the invention. An exploded adjustable nose splint 100 is illustrated along with its constituent parts, including: a first lateral splint 105, a second lateral splint 110, the hinge 115, the knurled nut 120, the threaded nuts 125, and the spring 130. The spring features two slots 135 into which the first and second lateral splints, 105 and 110, are inserted to attach the lateral splints, 105 and 110, to the spring.

What is claimed is:

1. An adjustable nose splint, comprising:
a first lateral splint, said first lateral splint being generally rigid and shaped to conform to the sidewall of a human nose,
a second lateral splint, said second lateral split being generally rigid and shaped to conform to the sidewall of the human nose and being arranged opposite the first lateral splint and jointedly attached to the first lateral splint by a hinge,
said hinge being configured to jointedly attach the first lateral splint to the second lateral splint such that the angle between the first lateral splint and the second lateral splint is variable,
a spring, said spring being attached to said first lateral splint and said second lateral splint such that said spring compresses said first lateral splint and said second lateral splint together, and
a threaded nut, said threaded nut being connected to said spring and configured such that turning said threaded nut allows the tension in said spring to be adjusted.

2. An adjustable nose splint according to claim 1, wherein said spring is removably attached to said first lateral splint and said second lateral splint.

3. An adjustable nose splint according to claim 1, wherein said spring is fixedly attached to said first lateral splint and said second lateral splint.

4. An adjustable nose splint according to claim 1, wherein said first lateral splint and said second lateral splint are generally triangular in shape.

5. An adjustable nose splint according to claim 1, wherein said first lateral splint and said second lateral splint feature concave edge sections that are generally configured to conform with the shape of the alar-sidewall.

6. An adjustable nose splint according to claim 1, wherein said first lateral splint and said second lateral splint feature curved sections adjacent to the hinge that are generally configured to conform with the shape of the nasal bone.

7. An adjustable nose splint according to claim 1, wherein said spring comprises an elastic material that is generally U-shaped.

8. An adjustable nose splint according to claim 1, wherein said spring further comprises a first threaded stud and a second threaded stud, said first threaded stud and said second threaded stud being configured to engage with said threaded nut such that the turning the threaded nut adjusts the tension in the spring.

9. An adjustable nose splint according to claim 1, wherein said first lateral splint and said second lateral splint further comprise padded areas on the interior of said first lateral splint and said second lateral splint.
